SEQUENCE LISTING (3 sequences)

SEQ ID NO: 1 - DNA, 20 bp, Artificial Sequence
  CpG Oligodeoxynucleotide
tccatgacgttcctgacgtt

SEQ ID NO: 2 - DNA, 32 bp, Artificial Sequence
  Sense primer with BamHI restriction site
ttgggatccaccatgcatggagatacacctac

SEQ ID NO: 3 - DNA, 24 bp, Artificial Sequence
  Antisense primer with EcoRI restriction site
cggaattcattcttatggtttctg

(19) United States
(12) Patent Application Publication
(10) Pub. No.: US 2004/0115219 A1
(43) Pub. Date: Jun. 17, 2004

(54) Pharmaceutical composition for prophylaxis and therapy of papillomavirus-derived diseases comprising papillomavirus antigen protein and CpG-oligodeoxynucleotide

(76) Inventors: Woong-Shick Ahn, Seoul (KR); Jeong-Im Sin, Daegu-si (KR)

Correspondence Address: Robert J. Ward, Esq., HUNTON & WILLIAMS, Energy Plaza, 30th Floor, 1601 Bryan Street, Dallas, TX, 75201-3402, US

(21) Appl. No.: 10/408,078

(22) Filed: Apr. 4, 2003

(30) Foreign Application Priority Data
Dec. 13, 2002 (KR) 2002-0079881

(51) Int. Cl. A61K 48/00; A61K 39/12

(52) U.S. Cl. 424/186.1; 514/44

(57) ABSTRACT

The present invention relates to a pharmaceutical composition for prophylaxis and therapy of papillomavirus-derived diseases which comprises papillomavirus antigen protein and CpG-oligodeoxynucleotide.

### CROSS-REFERENCE TO RELATED APPLICATIONS

Pursuant to 35 U.S.C. § 119(e), this application claims the benefit of Korean Patent Application No. 2002-0079881, entitled PHARMACEUTICAL COMPOSITION FOR PROPHYLAXIS AND THERAPY OF PAPILLOMAVIRUS-DERIVED DISEASES COMPRISING PAPILLOMAVIRUS ANTIGEN PROTEIN AND CpG-OLIGODEOXYNUCLEOTIDE, filed Dec. 13, 2002, and named Woog-Shick Ahn and Jeong-Im Sin as inventors, which is hereby incorporated by reference for all purposes.

### TECHNICAL FIELD OF THE INVENTION

This invention relates to a pharmaceutical composition for prophylaxis and therapy against papillomavirus-derived diseases which comprises papillomavirus antigen protein and CpG-oligodeoxynucleotide.

### BACKGROUND OF THE INVENTION

Papillomavirus (PV) has been known to cause severe diseases such as benign diseases, dysplasia and malignancy of the skin and epithelial regions [Mansur et al., Biochim Biophys Acta, 1155: 323-345, 1993; Pfister, Rev. Physiol. Biochem. Pharmacol., 99: 111-181, 1984; Broker et al., Cancer Cells, 4: 17-36, 1986].

Human papillomavirus (HPV) is an oncogenic DNA virus which is known to cause overgrowth of squamous epithelial cells and malignant lesions. Many women are infected with HPV by sexual contact and some of the infected women develop cervical cancer. Twenty (20)% of cancer-related deaths in women are due to cervical cancer.

HPV has been classified into two groups; a high risk group (types 16 and 18) and a low risk group (types 6 and 11) based upon the relative tendency of the lesion to progress to a cancer stage. HPV 16 infection is a major cause of cervical cancer worldwide [zur Hausen, H., J. Virol., 184: 9-13, 1991]. The expression of HPV oncogenic proteins, E6 and E7 is required for tumorigenesis and maintenance of the tumor state [Scheffner, M. et al., Proc. Nat'l. Acad. Sci. USA, 88: 5523-5527, 1991; Werness, B. A. et al., Science, 248: 76-79, 1990; Dyson, N. et al., Science, 243: 934-937, 1989]. In particular, the amino acid sequence of HPV type 16 E7 protein is derived from the code of its DNA sequence and is well described [N. Salzman and P. Hawley, “The Papoviridae”, Vol. 2, p. 379, Plenum Press, N.Y. (1987)].

Furthermore, E7-specific immune responses are detected in cervical cancer patients, suggesting that E7 could be a specific target for immunotherapy against HPV-derived cervical cancers [de Gruijl, T. D. et al., J. Gen. Virol., 77: 2183-2191, 1996]. In this regard, E7-specific prophylactic and therapeutic vaccine strategies have been evaluated in animal model systems. These include direct uses of recombinant E7 proteins [Fernando, G. J. P. et al., Clin. Exp. Immunol., 115, 1999], DNA vaccine encoding E7 [Hung, C. F. et al., Cancer Res., 61: 3698-3703, 2001], and bacterial/viral vectors expressing E7 or E7 epitope [Lamikanra, A. et al., J. Virol., 75: 9654-9664, 2001; Cheng, W. F. et al., Hum. Gene Ther., 13: 553-568, 2002; Liu, D. W. et al., J. Virol., 74: 2888-2894, 2000; Londono, L. P. et al., Vaccine, 14: 545-552, 1996], as well as CTL epitopes of E7 [Feltkamp, M. C. et al., Eur. J. Immunol., 23: 2242-2249, 1993]. In these studies, CD4+ T cell and in particular CTL activities have been correlated to protective immunity against tumor cells.

The immune system recognizes the DNA of low organisms including bacteria, probably due to structural and sequence differences between pathogen and host DNA. Specific interests focus on the short stretch of DNA derived from non-vertebrates or the DNA in the form of short oligodeoxynucleotides (ODNs) containing non-methylated cytosine-guanine dinucleotides. Recently, it has been found that bacterial DNA as such stimulates the immune response of mammals. The major difference between bacterial DNA and mammalian DNA is that bacterial DNA has a variety of CpG (cytosine-guanine) dinucleotides. Based on this, the synthetic CpG-ODNs including unmethylated CpG motifs have been used as immune stimulants.

Oligodeoxynucleotides containing unmethylated CpG motifs (CpG-ODN) can activate B cells, monocytes and NK cells, and induce Th1 like pattern of cytokine production [Bohle, B. et al., Eur. J. Immunol., 29: 2344-2353, 1999; Klinman, D. M. et al., Proc. Nat'l. Acad. Sci. USA, 93: 2879-2883, 1996; Krieg, A. M. et al., Nature, 374: 546-549, 1995; Ballas, Z. K. et al., J. Immunol., 157: 1840-1845, 1996; Sparwasser, T. et al., Eur. J. Immunol., 30: 3591-3597, 2000; Sparwasser, T. et al., Eur. J. Immunol., 28: 2045-2054, 1998]. In a number of animal studies, CpG motifs in bacterial DNA and synthetic ODNs are responsible for driving immune responses towards Th1 type responses [Chu, R. S. et al., J. Exp. Med., 186: 1623-1631, 1997; Leclerc, C. et al., Cell. Immunol., 179: 97-106, 1997; Klinman, D. M. et al., J. Immunol., 158: 3635-3639, 1997; Jakob, T. et al., J. Immunol., 161: 3042-3049, 1998]. The CpG sequences drive macrophages to secrete IL-12, a potent inducer of IFN-γ production in vivo from natural killer cells. IFN-γ production drives Th1 type immune responses by inducing the differentiation of type 1 T helper cells, which see antigen in the presence of IFN-γ from the uncommitted T cell pool [Chu, R. S. et al., J. Exp. Med., 186: 1623-1631, 1997; Roman, M. et al., Nature Med., 3: 849-854, 1997]. Moreover, ODN enhances humoral responses, driving them toward IgG2a isotypes (Th1 type indicator) [Chu, R. S. et al., J. Exp. Med., 186: 1623-1631, 1997; Davis, H. L. et al., J. Immunol., 160: 870-876, 1998] and induces the development of enhanced CTL activity [Krieg, A. M. et al., Nature, 374: 546-549, 1995; Warren, T. L. et al., J. Immunol., 165: 6244-6251, 2000]. ODNs have been extensively studied as strong immunomodulatory agents [Davis, H. L. et al., J. Immunol., 160: 870-876, 1998; Weiner, G. J. et al., Proc. Nat'l. Acad. Sci. USA, 94: 10833-10837, 1997; Davis, H. L. et al., Proc. Nat'l. Acad. Sci. USA, 93: 7213-7218, 1996; Kline, J. N. et al., J. Immunol., 160: 2555-2559, 1998; Scott Gallichan, W. et al., J. Immunol., 166: 3451-3457, 2001]. However, no studies on the effect of ODNs for immunotherapy against cervical cancer have been reported. Presently, many different approaches regarding the control of cervical cancer have been initiated with limited success. However, a therapy modality which can effectively control HPV-derived diseases is reported here.

In this invention, we observed that compared to an exclusive use of HPV antigenic protein E7 or CpG-ODN, a combination therapy using E7 and CpG-ODN is the only effective option for enhancing E7-specific antibody and Th1 type T cell responses, as well as for the induction of CTL responses and IFN-γ production from both CD4+ and CD8+ T cells. These cells are involved directly in mediating anti-cancer effects. Based on the above, this invention provides a powerful immunological method to selectively augment Th1 type CD4+ T cells and CTL, which eventually result in control of HPV-derived diseases.

### SUMMARY OF THE INVENTION

The present invention is related to a pharmaceutical composition for prophylaxis and therapy against papillomavirus-derived diseases, which comprises an immunologically effective amount of papillomavirus E7 antigen protein and CpG-oligodeoxynucleotide.

Preferably, the composition of this invention comprises the human papillomavirus type 16 E7 protein as the papillomavirus antigen protein.

More preferably, the composition of this invention comprises recombinant protein as the papillomavirus antigen protein.

Preferably, the composition of this invention comprises CpG-oligodeoxynucleotide which comprises 8 to 40 nucleotides with one or more CpG motifs in which one or more nucleotides separate continuous CpG motifs in the oligodeoxynucleotide.

More preferably, the composition of this invention comprises 5′-TCCATGACGTTCCTGACGTT-3′ as CpG-oligodeoxynucleotide.

Preferably, the composition of this invention is used for prophylaxis and therapy of cervical cancer.

### BRIEF DESCRIPTION OF THE DRAWINGS

FIG. 1 represents prophylactic effects of pharmaceutical compositions for injection, such as E7, CpG-ODN, and E7 plus CpG-ODN on tumor cell growth over time;

FIG. 2 represents therapeutic effects of pharmaceutical compositions for injection, such as E7, CpG-ODN, and E7 plus CpG-ODN on tumor cell growth over time;

FIG. 3 represents the effects of pharmaceutical compositions such as E7, CpG-ODN, and E7 plus CpG-ODN on the induction of E7-specific antibody responses (IgG, IgG1, IgG2a, IgG2b and IgG3);

FIG. 4 represents the effects of pharmaceutical compositions such as E7, CpG-ODN, and E7 plus CpG-ODN on the induction of E7-specific Th cell proliferative and CTL responses;

FIG. 5 represents the effects of pharmaceutical compositions such as E7, CpG-ODN, and E7 plus CPG-ODN on the production of IFN-γ from E7-specific CD4+ and CD8+ T cells; and

FIG. 6 represents the immune cell populations responsible for protective immunity against tumor cells in animals.

### DETAILED DESCRIPTION OF THE INVENTION

The present invention provides a pharmaceutical composition for prophylaxis and therapy against papillomavirus-derived diseases which comprises an immunologically effective amount of papillomavirus E7 antigen protein and CpG-oligodeoxynucleotide.

The term “immunologically effective amount” denotes that the amount administered to a papillomavirus-infected individual or an individual to be infected by the virus is effective for prophylaxis and therapy of papillomavirus-derived disease.

The term “papillomavirus-derived disease” denotes cell-proliferative disease of malignant or nonmalignant cell populations caused by papillomavirus, which morphologically often appear to differ from surrounding tissues. The papillomavirus may include all the pathogenic types, for example, type 16, 18, 32 and etc.

By use of the term “prophylaxis or therapy”, it is meant that the prophylaxis is to administer a drug before exposure to papillomavirus and the treatment is to administer a drug after infection or onset of the disease.

The term “antigen” denotes a molecule that can generate an immune response. For the purpose of this invention, the “papillomavirus antigen” is papillomavirus E7 antigen which is isolated from nature or prepared by recombination methods. In a preferred embodiment, the present invention uses human papillomavirus type 16 E7 protein as the papillomavirus E7 antigen protein.

The papillomavirus antigen protein, which can be comprised in a pharmaceutical composition of this invention and isolated from nature or prepared by recombination methods, denotes the protein that has the sequence of natural protein as well as 85% or more, preferably 90% or more, of sequence homology and induces the substantially same immune response as that of the natural papillomavirus antigen protein. The protein isolated from nature can be isolated and purified from the large scale culture of papillomavirus by the method known in the art.

The particularly preferred papillomavirus antigen protein of this invention is the recombination E7 protein of human papillomavirus type 16, which is produced by the genetic recombination method.

E7 recombinant protein used in this invention can be prepared by various recombination expression vectors known in the art.

The term “vector” denotes the DNA constructs comprising DNA sequences that are operatively connected to suitable regulatory sequences, which can express DNAs in host cells. For the purpose of this invention, any one of the recombination expression vectors can be used as long as they can express papillomavirus antigen proteins. For example, plasmid, phage, other viruses, etc. can be used for this invention. Generally, when a suitable host is transformed by a recombinant vector, the vector can replicate and function without any reliance to host genomes, and in some cases, the vector can be integrated into the host genome.

The expression vectors, which are suitable for the expression of papillomavirus antigen protein and can be used in eukaryote hosts are, for example, SV40, retrovirus, adenovirus, herpes simplex virus, poxvirus, lentivirus, adeno-associated virus, cytomegalovirus, etc.; the vectors that can be used in bacterial hosts are, for example, bacterial plasmids originated from Escherichia coli such as pBluescript, pMAL-c2x, pGEX2T, pUC, pCR1, pBR322, pMB9 and their derivatives, etc., RP4 which has a broad host range, DNA phage such as λgt10, λgt11, etc., and other DNA phages including the DNA phage that is a filamentous single strand such as M13. Expression vectors useful in yeast cells include 2 μm plasmid and its derivatives, and the vectors useful in insect cells include pVL 941, etc.

Generally, the recombination expression vectors include expression regulatory sequences, which are essential for the expression of coding sequences in hosts and operatively connected to them. To express the DNA sequence according to this invention, a variety of expression regulatory sequences can be used. For example, promoter sequences for transcription, operator sequences for the regulation of transcription, mRNA ribosomal binding site-coding sequences, and regulatory sequences for the termination of transcription and translation can be included. For example, the regulatory sequences suitable for prokaryotes include promoter, operator, ribosome binding site, etc. The regulatory sequences suitable for expression in eukaryotes include promoter, polyadenylation signal, enhancer, etc. The factor that significantly influences the amount of expression is the promoter and preferably SRαpromoter, and the cytomegalovirus-originated promoter is used as a high-expression promoter.

Furthermore, a nucleic acid sequence can be operatively connected to the other nucleic acid sequence(s). For example, the sequence coding for transcription activating protein, which allows the expression of genes is connected to the regulatory sequence(s); pre-sequence or secretion leader coding sequence is connected to the nucleic acid sequence coding for the interested protein or peptide; promoter or enhancer sequence that influences transcription is connected to the sequence coding for the interested protein or peptide; or the ribosomal binding site, which influences transcription is connected to the sequence coding for the interested protein or peptide.

The recombination vector of this invention can be introduced into cells by conventional transformation methods, for example, the DEAE-dextran method, the calcium phosphate method, the electroporation method, etc. The techniques for the transformation of host and the expression of the cloned foreign DNA sequence in the host have been well known in this art [Maniatis et al., Molecula Cloning: A Laboratory Manual, Cold Spring Harbor Laboratory (1982); Gene Expression Technology, Method in Enzymology, Genetics and Molecular Biology, Methods in Enzymology, Guthrie & Fink (eds.), Academic Press, San Diego, Calif., 1991; Hitzeman et al., J. Biol. Chem., 255: 12073-12080, 1980].

The protein used in this invention can be expressed as a fusion protein, in which the desired protein is fused to the other protein. For example, a fusion protein of E7 protein and glutathione-S-transferase (GST) can be expressed in Escherichia coli [Fernando G. J., Clin. Exp. Immunol, 115(3): 397-403, 1999 March]. In addition, a fusion protein may be prepared by the transfection of host cells with adeno-associated virus (AAV) comprising the gene cording for E7 protein fused to the gene cording for heat shock protein [Liu D. W. et al., J. Virol., 74(6): 2888-94, 2000 March].

The papillomavirus antigen protein may be glycosylated, lipidated, or derivatized to comprise the molecules, which enhance antigen presentation or the targeting of antigen to the antigen presentation cell.

The papillomavirus antigen protein is preferably used in isolated and purified forms. For this, common techniques to purify proteins may be used. In this connection, to facilitate the separation and purification of papillomavirus recombinant protein, the protein may be prepared as a fusion protein with GST, His-tag, etc.

The E7 recombinant protein prepared in this invention has 98 amino acids. The protein is produced by expressing plasmid vector pET-E7 in Escherichia coli as fusion proteins of E7 protein and His-tag peptide of pET vector, in which His-tag peptide residue facilitates the purification of proteins. The purified E7 recombinant protein is preferably used after eliminating endotoxins from the protein.

Further, the natural or recombinant papillomavirus antigen protein comprised in the composition of this invention may be modified by common techniques in the art.

The term “CpG-oligodeoxynucleotide(CpG-ODN)” denotes the oligonucleotides which comprise 8 to 40 nucleotides with one or more CpG(cytosine-phosphorothioate-guanine) motifs in which one or more nucleotides separate continuous CpG motifs in the oligodeoxynucleotide.

Preferably, continuous CpG dinucleotides are separated by one or more nucleotides.

More preferably, the oligodeoxynucleotide includes 5′-TCCATGACGTTCCTGACGTT-3′ (SEQUENCE ID NO 1).

The CpG-ODN may be chemically synthesized, recombinantly constructed, or derived from natural sources. Of course, mixtures of different CpG-ODNs may be used.

Chemically synthesized CpG-ODN may be synthesized de novo using various methods known in the art. For example, β-cyanomethyl phosphoramidate method [S. L. Beaucage et al., Tet. Let., 22: 1859, 1981], nucleoside H-phosphonate method [Garegg et al., Tet. Let., 27: 4051-4054, 1986; Froehler et al., Nucl. Acid. Res., 14: 5399-5407; Garegg et al., Tet. Let. 29: 2619-2622, 1988], etc. may be utilized. The chemical synthetic method may be carried out by using various automatic oligonucleotide synthesizers. Alternatively, the oligonucleotide may be prepared from nucleic acids (for example, genome or cDNA) using restriction enzyme, exonuclease or endonuclease.

Further, CPG-ODN may be suitably modified in order to resist degradation in vivo. Preferably, the modification includes a phosphorothioate modification. The phosphorothioate modification may occur at either terminus: for example, the last two or three 5′ or 3′ nucleotide may be linked with phosphorothioate bonds. The CpG-ODN can also be modified to contain a secondary structure (for example, a stem loop structure) such that it is resistant to degradation. Preferably, stabilized nucleic acid may have one or more partially phosphorothioate-modified backbone. The phosphorothioate may be synthesized by automatic techniques using phosphoroamidate or H-phosphonate chemistry. Aryl- and alkyl-phosphonate can be prepared, for example, as described in U.S. Pat. No. 4,469,863, and akylphosphotriester(i.e., the charged phosphonate oxygen is alkylated as set forth in U.S. Pat. No. 5,023,243 and European Patent No. 0 092 574) can be prepared by automatic solid phase synthesis using a commercial reagent. Methods for the preparation of DNA backbone modification or for the substitution are disclosed in the literatures [Uhlmann, E. et al., Chem. Rev. 90: 544, 1990; Goodchild, J., Bioconjugate Chem., 1: 165, 1990]. Another modification that renders the ODN less susceptible to degradation is the inclusion of nontraditional bases such as inosine and quesine as well as acetyl-, thio- and similarly modified forms of adenine, cytidine, guanine, thymine, and uridine. ODNs containing a diol, such as tetraethyleneglycol or hexaethyleneglycol, at either or both termini, have also been shown to be more resistant to degradation.

When administered in vivo, CpG-ODN may form a “nucleic acid transfer complex”, which is linked to a molecule for high affinity bond to the surface of target cells or increased uptake of cells. Nucleic acids can be linked to any suitable molecules by ionic or covalent bonds using the techniques well known in the art. Suitable coupling or cross-linking agents such as Protein A, carbodiimide, N-succinimidyl-3-(2-pyridyldithio)propionate(SPDP), etc. can be used. CpG-ODN can be also capsulated in liposome or virosome using the techniques well known in the art.

In the present invention, the anti-tumor effect was observed in animal models by co-immunization of E7 protein and CPG-ODN. E7-specific antibody response, Th cell proliferative response, CTL response and IFN-γ production by CD4+ T lymphocyte and CD8+ T lymphocyte were also observed.

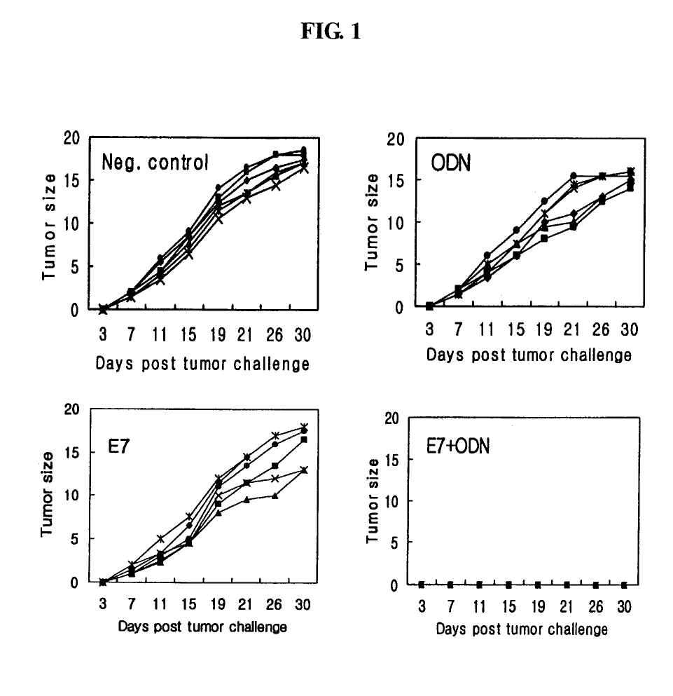

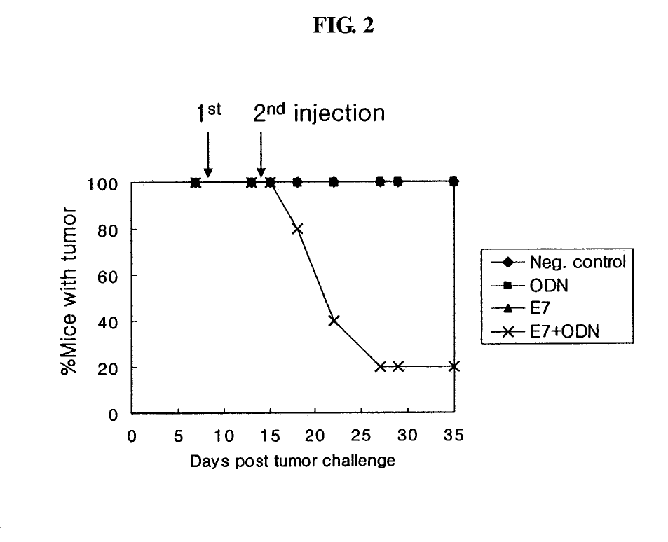

Specifically, anti-tumor effects were observed in the E7 expression tumor cell line by co-immunization of E7 protein and CpG-ODN (FIG. 1 and Table 2). When E7 protein or CpG-ODN was injected alone, the prophylactic effect of anti-tumor was not observed as shown in FIG. 1 and Table 2. Only when both the E7 protein and the CpG-ODN were injected, the protective effect was observed. Similarly, the therapeutic effect of anti-tumor was observed only when both the E7 protein and the CPG-ODN were co-injected (FIG. 2). This shows that both the papillomavirus antigen protein and the CPG-ODN are essential for the prophylatic and therapeutic effect against papillomavirus-derived disease.

As shown in the above, in view of the fact that the anti-tumor effect could not be observed when the papillomavirus antigen protein or the CpG-ODN was used exclusively, but the effect could be observed only when both of them were used, the anti-tumor effect of the present invention could never have been expected from the previous art that CpG-ODN was used in itself or as an adjuvant to enhance the known immune effect.

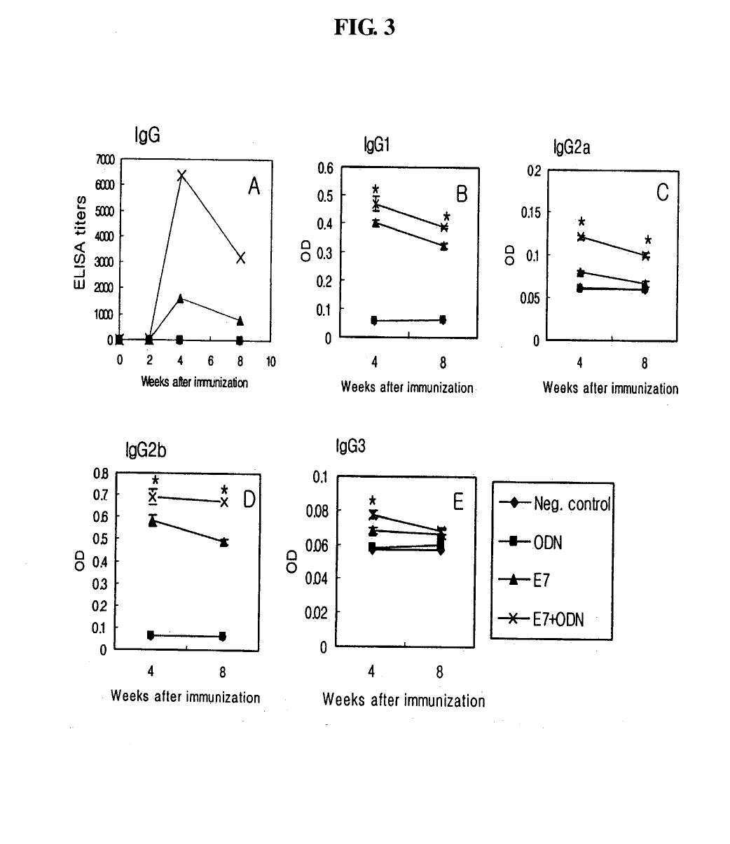

Further, as a result of the observation of E7-specific antibody response by co-injection of E7 protein and CPG-ODN, when E7 protein was injected together with CpG-ODN, the ELISA titer was higher than E7 protein alone, showing a more strengthened antibody response (FIG. 3A), and also the production of IgG isotypes, i.e., IgG1, IgG2a, IgG2b and IgG3 was more increased (FIGS. 3B, C, D and E).

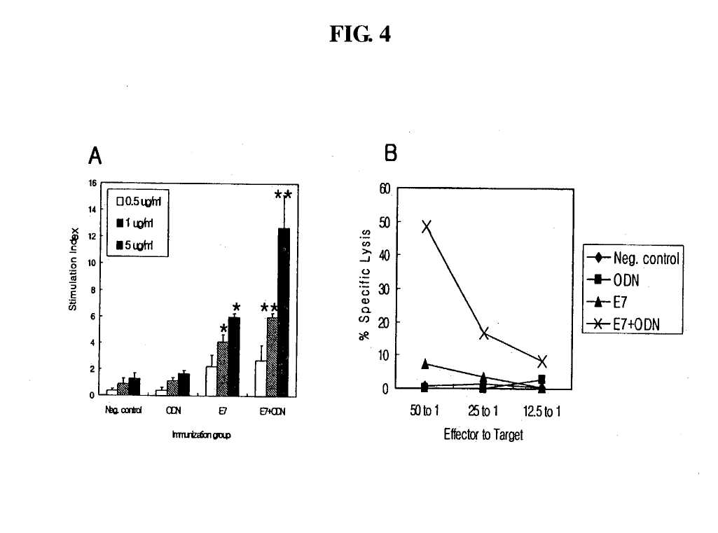

For Th cell proliferative response by co-injection of E7 protein and CpG-ODN, a significant enhancement of Th cell proliferative response was observed compared to the injection of E7 protein or CpG-ODN alone (FIG. 4A). When CD4+ T lymphocytes were removed, the Th cell proliferative response was never detected, showing that the antibody response directly relates to Th cell proliferative response in association with the CD+4 T lymphocyte.

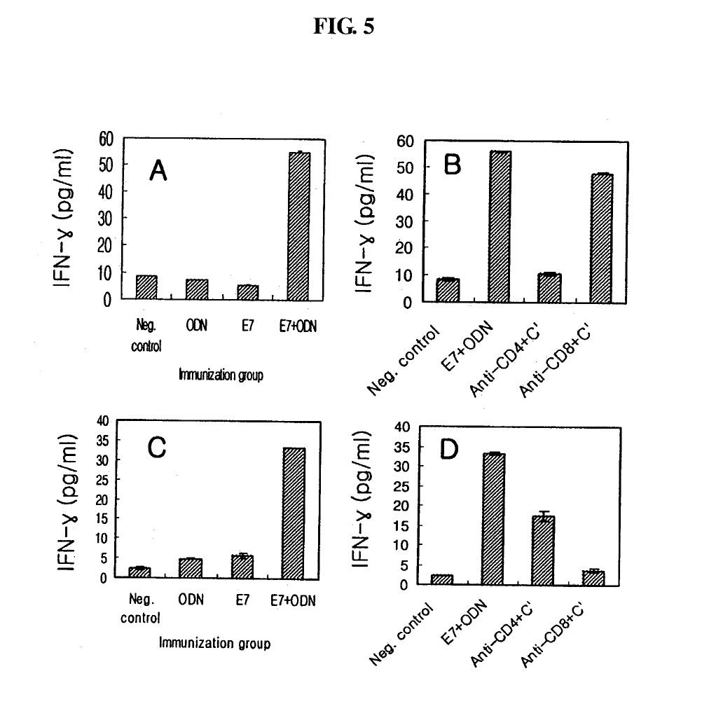

Further, only when E7 protein and CpG-ODN were co-injected, the CTL response was observed, but when E7 or CpG-ODN was injected alone, the response was not observed (FIG. 4B). This result suggests the essential role of CpG-ODN on the induction of antigen-specific CTL response by E7 protein. Further, the production of IFN-γ by the co-injection of E7 protein and ODN was evaluated. Although the effect of CPG-ODN in the production of IFN-γ was reported [Chu, R. S. et al., J. Exp. Med., 186: 1623-1631, 1997], the production of IFN-γ by CD4+T lymphocyte was induced only in the animal co-injected with E7 protein and CPG-ODN (FIG. 5B). This suggests that when stimulated with E7 antigen, CD4+ T lymphocytes, not CD8+ T lymphocytes, produce IFN-γ. Similarly, when stimulated with TC-1 cell line, the production of IFN-γ from CD8+ lymphocytes was observed only in the animals co-injected with E7 protein and CpG-ODN, whereas the production of IFN-γ was not observed in the animals injected with E7 or CPG-ODN alone (FIG. 5D). This suggests that when stimulated with TC-1 cell line, CD8+ T lymphocytes, not CD+4 T lymphocytes, secrete IFN-γ.

This result is consistent with that of the CTL reaction induced when co-injected with E7 protein and CPG-ODN. This suggests that both E7 protein and CPG-ODN are required for the induction of IFN-γ production in CD+4 and CD8+ T lymphocytes in concert with MHC I and II. In this connection, the protective immunity of IFN-γ to virus infection or anti-tumor has been reported [Samuel, C. E., J. Virol., 183: 1-11, 1991; Smith, P. M. et al., Virol., 202: 76-88, 1994; Boehm, U. et al., Annu. Rev. Immunol., 15: 749-795, 1997; Yang, Y. et al., Proc. Nat∝l. Acad. Sci. USA, 89: 4928-4932, 1992].

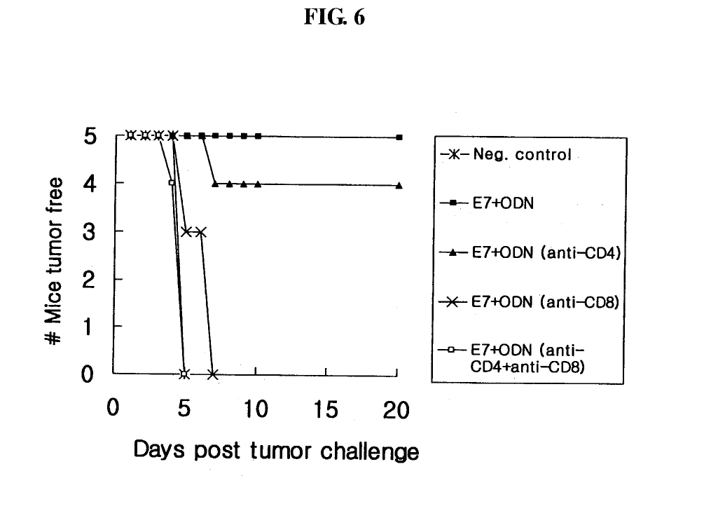

The role of CD4+ and CD8+ T lymphocytes to the anti-tumor immune response against TC-1 cell line was evaluated (FIG. 6). When both CD4+ and CD8+ T lymphocytes were depleted from the animal co-injected with E7 protein and CPG-ODN, the result is similar with that of the non-treated control group. Meanwhile, when only CD8+ T lymphocyte was depleted, the animals showed a bit delayed, but a completed formation of tumor. This proves the major role of CD8+ T lymphocytes in protection against tumor formation. When depleted of CD4+ T lymphocytes, tumor growth was observed in 4 out of 5 animals, showing that CD4+ T lymphocytes are also the immune cell populations contributing to protective immunity. This suggests that CD4+ and particularly CD8+ T lymphocytes show an anti-tumor effect. However, antibodies did not contribute to the anti-tumor effect against TC-1.

This result is in concert with other results showing that CD4+ or CD8+ effector T lymphocyte populations have anti-tumor activity against the TC-1 tumor cell line [Hung, C. F. et al., Cancer Res., 61: 3698-3703, 2001; Lamikanra, A. et al., J. Virol., 75: 9654-9664, 2001; Cheng, W. F. et al., Hum. Gene Ther., 13: 553-568, 2002; Liu, D. W. et al., J. Virol., 74: 2888-2894, 2000; Lin, K. Y. et al., Cancer Res., 56: 21-26, 1996].

TABLE 1
Immune responses and protection against tumor challenge

| | | Th | CD4+ | | Prophy- | Thera- |
|---|---|---|---|---|---|---|
| | Antibody | proliferative | T | CTL | lactic | peutic |
| | responses | responses | (Th1) | (CD8) | effects | effects |
| Control | − | − | − | − | − | − |
| E7 | ++ | ++ | − | − | − | |
| CpG− | − | − | − | − | − | − |
| ODN | | | | | | |
| E7 + | +++ | +++ | +++ | +++ | +++ | +++ |
| CpG− | | | | | | |
| ODN | | | | | | |

+ represents stronger responses;
− represents no responses.

As obviously shown in the above, CpG-ODN acts as an adjuvant that enhances E7 antigen-specific response, Th proliferative response, and activity of CD4+ T lymphocyte secreting IFN-γ(Th1) and CD8+ T lymphocyte secreting IFN-γ(CTL), proving that immunization with both E7 protein and ODN is useful for the induction of antigen-specific Th1 type CD4+ and in most part CD8+ T cell immune responses, and thus results in the superior immune effect controlling the papillomavirus-induced diseases, particularly HPV-associated cervical cancer.

The pharmaceutical composition of this invention can be prophylatically or therapeutically administered to the subject who is infected or is suspected of being infected with papillomavirus. The subject administered includes patients that suffer from papillomavirus-induced diseases or will suffer from the disease. The diseases include, for example, bowenoid papulosis, anal dysplasia, respiratory or conjunctival papillomas, cervical dysplasia, cervical cancer, vulval cancer, prostate cancer and the like.

Particularly, the pharmaceutical composition of this invention is useful for the prevention and treatment of cervical cancer induced by papillomavirus.

The pharmaceutical composition may be administered as such or with any other means known in the art such chemotherapy, radiation therapy, surgical operation, etc. Also, other adjuvants or cytokines can also be administered. The cytokines, which can be co-administered to stimulate immune response, include granulocyte-macrophage colony stimulating factor (GM-CSF), granulocyte colony stimulating factor(GCSF), IL-1, IL-2, IL-3, IL-4, IL-5, IL-6, IL-7, IL-10, IL-12, IL-15, TNF-α, TNF-γ, Flt3 ligand, etc.

The pharmaceutical composition of this invention can be administered by the methods well known in the art [Donnelly et al., J. Imm. Methods, 176: 145, 1994; Vitiello et al., J. Clin. Invest., 95:341, 1995]. The preparations administered include tablets, troches, dispersions, suspensions, solutions, capsules, creams, ointments, suppositories, aerosols, etc. The preparation also includes implanted slow releasing devices, etc. The preparation can be administered by the methods known in the art, for example, orally or parenternally such as intramusclely, intraveneously, intraarterially, intradermally, intraperitoneally, intranasally, intravaginally, intrarectally, sublingually or subcutaneously, as well as into the gastrointestinal track, the mucosa or the respiratory track.

The pharmaceutical composition of this invention can be administered topically or systemically. Topical administration is advantageous so as to localize the drug in the site administered, with minimized systemic uptake. When administered topically, smaller dosages than other administration routes can be administered. The preparations for topical administration include transdermal devices, aerosols, creams, lotions, powders, etc.

The pharmaceutical composition can be formulated with one or more pharmaceutically acceptable carrier or optional adjuvants that facilitate the formulation, including excipients. The formulation depends on the administration route. For injection, the active ingredient can be formulated into aqueous solutions, preferably in a saline solution. For transmucosal administration, penetrants appropriate to the barrier to be permeated are used in the formulation. Such penetrants are generally known in the art. For oral administration, the active ingredient can be combined with carriers suitable for inclusion into tablets, pills, dragees, capsules, liquids, gels, syrups, slurries, suspensions and the like. For administration by inhalation, the active ingredient is conveniently delivered in the form of an aerosol spray presentation from pressurized packs or a nebuliser with the use of a suitable propellant or the form of a powder, which can be formulated into cartridges. Also, when administered by injection, the active ingredient can be formulated into forms such as suspensions, solutions, emulsions, etc.

The composition can be administered at a dosage of about 0.1 μg/kg/day to about 3 μg/kg/day, preferably 0.5 μg/kg/day to about 1 μg/kg/day at an interval of at least a week. It depends on various factors such as weight, age, sex, administration route, formulation, time, and the general health condition, etc of individuals.

The present invention is further illustrated by the following examples. But, such examples are expressly incorporated for the description of the present invention and should not be construed as further limiting of this invention.

### EXAMPLES

Abbreviations ODN, oligodeoxynucleotide; HPV, human papillomavirus; PBS, phosphate-buffered saline; HRP, horse radish peroxidase; HSV, herpes simplex virus; OD, optical density; IPTG, isopropyl-β-D-thiogalactopyranoside; RT-PCR, reverse transcription-polymerase chain reaction; i.p., intraperitoneally; s.c., subcutaneously; SI, stimulation index.

A paired Student's T test was performed for statistical analysis. The p values less than 0.05 were considered statistically significant.

### Example 1

Production of Recombinant E7 Proteins

The recombinant E7 protein was expressed and purified as described [Protocols of Novagen Inc. and Sin, J. I. et al., Vaccine 15: 1827-1833, 1997].

The HPV type 16 E7 gene was amplified by reverse transcription-polymerase chain reaction (RT-PCR) from a Caski cell line with a pair of primers: the Bam HI containing sense primer, 5′-TTGGGATCCACCATGCATGGAGATACACCTAC-3′ (SEQUENCE ID NO 2) and Eco RI-containing anti-sense primer, 5′-CGGAATTCATTCTTATGGTTTCTG-3′ (SEQUENCE ID NO 3). The amplified DNA was digested with BamHI and EcoRI and the resulting DNA fragment was gel purified. The E7 DNA fragments were then cloned into the BamHI and EcoRI site of the pET vector (Novagen, Madison, Wis.). The plasmid construct was transformed into E. coli DH5 and selected against kanamycin. The pET-E7 vector was purified and again transformed into E. coli BL21(DE3) cells and incubated in LB broth supplemented with kanamycin at a final concentration of 30 μg/ml. The cells were incubated in a shaker until absorbance at 600 nm was between 0.6 and 0.8 optical density (OD) units. Proteins were induced by addition of 1 mM isopropyl β-D-thiogalactopyranoside (IPTG) for 3 h. The cell pellets were collected at 4 krpm for 20 min and frozen-thawed once at −20□. The cell pellet was resuspended in 5 ml of 8 M urea buffer (pH 8.0) per gram wet weight. The cells were lysed by stirring for 15-60 min at room temperature and centrifuged at 1.5 krpm for 30 min. The cell supernatants were collected and passed through for the Ni-NTA resin column (Qiagen, Valencia, Calif.) pre-equilibrated with 8 M urea buffer (pH 8.0). The resin was washed with 5 vol. of Buffer B (8 M urea buffer, pH 8.0) and then with 5-10 vol. of Buffer C (8 M urea buffer, pH 6.3). In the final step, His-tagged E7 protein was eluted with 10 ml of Buffer C containing 200 mM imidazole. The protein solution was then dialyzed in 6 M urea buffers and then in 4 M urea buffers at 2 h intervals. This was followed by an overnight dialysis in a phosphate-buffered saline (PBS). The protein solution was collected and passed through the Detoxi-Gel endotoxin removing gel column (Pierce, Rockford, Ill.) according to the manufacturer's protocol except for the final elution with PBS. The protein concentration was calculated by the Bradford procedure using bovine serum albumin as a standard [Bradford, M. M., Anal. Biochem., 72: 248-254, 1976]. The endotoxin level of the E7 recombinant protein was checked using the endotoxin detection kit (Sigma, Saint Louis, Mo.). The final protein solution was stored at −70° C.

Protein samples were separated on 12% sodium dodecyl sulfate (SDS) polyacrylamide gel. The proteins were electrophoretically transferred to nitrocellulose membranes (Amersham, Piscataway, N.J.). The membrane was pre-equilibrated with TBST solution [10 mM Tris-HCl (pH 8.0), 150 mM NaCl, 0.1% Tween 20] containing 2% bovine serum albumin for 1 h and then reacted with anti-E7 monoclonal antibodies (Oncogene, Boston, Mass.) for 1 h at room temperature. After three washes with TBST, the membrane was incubated with anti-mouse IgG-horseradish peroxidase (HRP) (Sigma) for 1 h at room temperature. The immunoreactive protein bands were visualized using the ECL detection reagents (Amersham).

The recombinant E7 protein containing at least 98 amino acids of HPV 16 types was expressed in E. coli. The recombinant E7 protein migrated as a 23 kD protein in SDS-PAGE and was reactive to the HPV 16 E7 monoclonal antibodies. A molecular mass of the 23 kDa protein was larger in size than predicted (11 kD of E7 protein plus 4 kD protein of His-tagged regions in the pET vector system). This abnormal migration pattern of E7 protein was previously reported [Armstrong, D. J. et al., Biochem. Biophys. Res. Commun., 192: 1380-1387, 1993; Fernando, G. J. P. et al., Clin. Exp. Immunol., 115: 397-403, 1999].

Endotoxin levels of the recombinant protein were determined to be less than 100 EU/mg, as determined by the Endotoxin detection kit (Sigma, Saint Louis, Mo.).

### Example 2

Construction of CPG-ODN

The immunostimulatory CpG-ODN designated as 1826 (5′-TCCATGACGTTCCTGACGTT-3′) was used as a vaccine adjuvant in this study. The ODN was purchased from Biobasic Inc., Canada. ODN was synthesized with a nuclease-resistant phosphorothioate backbone. ODN was dissolved in water and was confirmed to have an undetectable endotoxin level.

### Example 3

Anti-Tumor Activity

Female 4-6 week old C57BL/6 mice were purchased from Daehan Biolink, Korea. Mice were injected subcutaneously (s.c.) with 20 μg of recombinant E7 protein and/or 20 μg of ODN in a final volume of 100 μl of PBS using a 28-gauge needle (Becton Dickinson, Franklin Lakes, N.J.).

TC-1 tumor cells (a kind gift from T. -C. Wu, Johns Hopkins Medical Institutions) were grown in cRPMI supplemented with 400 μg per ml of G418. TC-1 is an E7-expressing tumorigenic cell line. It was established from primary lung epithelial cells of C57BL/6 mice immortalized with HPV 16 E6 and E7 and then transformed with an activated ras oncogene [Wu, T. C. et al., Proc. Nat'l. Acad. Sci. USA, 92: 11671-11675, 1995]. 2×105 and 5×104 TC-1 cells were injected s.c. into the right flank of C57BL/6 mice for prophylactic and therapeutic vaccine studies, respectively. These challenge doses were previously tested [Lin, K. Y. et al., Cancer Res., 56: 21-26, 1996]. The tumor cells were washed 2 times with PBS and injected into mice.

### Example 3.1

Prophylactic Efficacy Against Tumor

Prophylactic efficacy of immunizations with E7, CpG-ODN and E7+CpG-ODN was evaluated against tumor challenge using the above mentioned animal model system.

FIG. 1 and Table 2 represent anti-tumor protective effects of E7, CpG-ODN and E7+CpG-ODN. Each group of mice (n=11 or 16) were injected subcutaneously (s.c.) with 20 μg of recombinant E7 protein and/or 20 μg of ODN at 0 and 2 weeks. Three weeks after the second injection, animals were challenged with 2×105 of TC-1 cells.

TABLE 2
Anti-tumor protective activities

| Immunization | Days after tumor challenge (day) | | |
|---|---|---|---|
| group | 15 | 25 | 45 |
| Control | 16/16(100)* | 16/16(100)* | — |
| CpG-ODN | 11/11(100)* | 11/11(100)* | — |
| E7 | 16/16(100)* | 16/16(100)* | — |
| E7 plus CpG- | 0/11(0)* | 0/11(0)* | 0/11(0)* |
| ODN | | | |

*# animals showing a tumor/# animals challenged with tumor cells (%)

As shown in Table 2, E7+ODN injection alone resulted in complete protection from tumor challenge, whereas E7 or ODN vaccine alone showed 100% tumor formation in animals in a manner similar to negative controls. Conversely, no protective efficacy was observed by immunization with either E7 or CpG-ODN alone. However, complete protection against tumor challenge was observed by immunization with both E7 and CpG-ODN.

FIG. 1 shows one representative experiment. Mice given injections of TC-1 cells developed rapidly growing tumors at the site of injection in negative control, E7, and ODN immunized animals over time. However, no tumor growth was observed in mice given injections of E7+ODN. In particular, E7 or ODN injected groups as well as negative group animals were all dead within 30-40 days post tumor challenge, while E7+ODN group survived far longer than 45 days (more than 4 months). Postmortem autopsy confirmed that death was due to cachectic shock and showed no signs of metastasis to other organs.

These data demonstrate that ODN as a vaccine adjuvant can induce complete protection against TC-1 tumor challenge.

### Example 3.2

Therapeutic Efficacy

For therapeutic studies, 5×104 TC-1 cells were injected s.c. into the right flank of C57BL/6 mice. When tumor size reaches about 1-2 mm in diameter, each group of mice was injected s.c. into the distal site of tumor injection with 20 pg of E7, 20 μg of CpG-ODN or 20 μg of E7 plus 20 μg of CpG-ODN, and then re-injected 1 week after the first injection. Mice were monitored twice per week for tumor growth. Tumor growth was measured in mm using a caliper, and was recorded as mean diameter [longest surface length (a) and width (b), (a+b)/2].

FIG. 2 represents therapeutic efficacy of E7+CpG-ODN against an established tumor. The animal groups injected with E7+ODN had 20% pf the animals exhibiting tumor on the flank. In contrast, mice immunized with E7 or ODN alone showed 100% of the animals with tumors, similar to negative control. Mice showing about 1-2 mm in tumor size developed rapidly growing tumors at the site of injection over time when immunized with E7 or ODN. However, tumor growth was suppressed completely in mice given injections of E7+ODN with the exception of two of the ten. These two mice displayed a more slowly growing tumor, as compared to other control groups. Furthermore, E7 or ODN injected groups as well as negative control animals were all dead within 40 days post tumor challenge while E7+ODN-injected animals survived far longer than 2 months.

This supports that E7+ODN co-injection can induce the suppression of an established tumor.

Example 3.3

Specific Roles of Immune Cell Populations

FACS analysis was used to count CD4+ and CD8+ T cells. Animals were immunized s.c. with E7 and/or CPG-ODN. Animals were sacrificed and spleen was obtained. Spleen cells (1×105) were washed 3 times with FACS buffer (PBS+1% BSA+0.1% sodium azide) and then reacted with phyco-erythrin conjugated anti-mouse CD4 and CD8 (Pharmingen, San Diego, Calif.) for 30 min on ice. After washing 3 times with FACS buffer, cells were analyzed for the percentage of CD4 or CD8 positive cells on a flow cytometer (Coulter-Epics XL, Miami, Fla.).

In vitro and in vivo depletion of CD4+ and CD8+ T cells were performed as previously described [Sin, J. I. et al., Human Gene Therapy, 12: 1091-1102, 2001; Sin, J. I. et al., J. Immunol., 162: 2912-2921, 1999].

For in vitro cell depletion, splenocytes were reacted with anti-CD4 (Pharmingen) or anti-CD8 (Accurate Chemical & Scientific Corp., Westbury, N.Y.) for 1 h at 4° C., followed by incubation with rabbit complement (Sigma) for 1 h at 37° C. Cell viability postdepletion was determined by trypan blue dye exclusion. Two cycles of antibodies plus complements resulted in depletion of more than 98% specific T cell subpopulation by FACS analysis.

For in vivo cell depletion, anti-CD4 (clone GK1.5) and anti-CD8 (clone 2.43) ascites fluids were generated by injecting hybridoma cells (American Type Culture Collection, Manassas, Va.) into pristane-primed nude mice intraperitoneally (i.p.). 100 μl of ascites fluids were administered i.p. on days −3, 0 and 3 of the tumor challenge. Antibody treatment resulted in more than 98% depletion of specific CD4+ and CD8+ T cell subsets of representative animals over a 3 week period. Depleted mice were subsequently challenged with tumor on day 0.

FIG. 6 represents the roles of CD4+ or CD8+ T cells in E7+ODN-induced protective immunity against challenge with E7-expressing TC-1 tumor cells. As shown in FIG. 6, following E7+ODN vaccination, we depleted CD4+ T cells, CD8+ T cells, or both in vivo and then tested the effects of specific cell populations on tumor protection. Each group of mice (n=5) was immunized s.c. with 20 μg of E7 and/or 20 μg of CpG-ODN at 0 and 2 weeks. At 3 weeks after the second injection, animals were depleted of CD4+ T cells, CD8+ T cells, or both, followed by s.c. challenge with 2×105 TC-1 cells. Animals showing no tumor growth were then counted.

As previously observed in FIG. 1 and Table 1, co-injection with E7 and CpG-ODN resulted in complete suppression of tumor growth when animals were not depleted of T cells in vivo. However, animals depleted of both CD4+ and CD8+ T cells failed to protect tumor growth in a manner similar to negative control animals. In particular, CD8+ T cell-depleted animal group showed a bit delayed, but a complete formation of tumor, as compared to a negative control group or the animal group depleted of both CD4+ and CD8+ T cells, suggesting a contributing role of CD4+ T cells and a major role of CD8+ T cells in protection against tumor formation. Moreover, animals depleted of CD4+ T cells protected tumor growth in 4 out of 5 animals. In particular, the remaining one CD4+ T cell depleted animal displayed a far smaller tumor than other groups with tumors over the time periods (data not shown).

These data support that E7 in the presence of ODN can induce protection from tumor growth through effects on CD4+ T cells and in most part CD8+ T cells in vivo.

### Example 4

Induction of Immune Responses

### Example 4.1

Induction of Antibody Responses

Enzyme linked immunosorbent assay (ELISA) was performed as previously described (Sin, J. I. et al., Vaccine, 15: 1827-1833, 1997; Sin, J. I. et al., J. Virol., 74: 11173-11180, 2000].

Each group of mice (n=10) was immunized s.c. with 20 μg of E7 and/or 20 μg of ODN at 0 and 2 weeks. Mice were bled at 2, 4 and 8 weeks following the first injection. For ELISA, the recombinant E7 protein (1 μg/ml in PBS) was used as a coating antigen. For the determination of relative levels of E7-specific IgG subclasses, anti-murine IgG1, IgG2a, IgG2b, or IgG3 conjugated with HRP (Zymed, San Francisco, Calif.) were substituted for anti-murine IgG-HRP. To determine ELISA titers, sera pooled in an equal volume from 10 mice per group were twofold serially diluted and reacted with E7 protein. The titers were determined as the reciprocals of the highest serum dilutions showing optical density values twice as high as that of the negative control.

In ELISA titers, equally pooled 2, 4 and 8 week sera were serially diluted and reacted with E7 to determine ELISA titers (FIG. 3A). Moreover, equally pooled 4 and 8 week sera per group were diluted to 1:100 and reacted with E7 protein in ELISA (B-E). Optical density was measured at 405 nm. *Statistically significant at P<0.05 using Student's T test compared to E7 alone.

FIG. 3 represents the induction levels of antigen-specific IgG in animals immunized with E7, CpG-ODN and E7 plus CpG-ODN. As shown in FIG. 3A, ELISA titers of equally pooled sera collected 2 weeks post the second immunization were determined as 1,600 (E7) and 6,400 (E7+ODN), a twofold increase in titer. Similarly, those of sera collected 6 weeks after the second immunization were determined to be 800 (E7) and 3,200 (E7+ODN). However, little induction of antibody titer was observed in ODN-injected groups similar to negative control.

FIGS. 3B, C, D and E represent the induction levels of antigen-specific IgG subtypes by different immunization protocols. E7+ODN vaccination enhanced all four types of IgG isotype production significantly higher than E7 vaccination alone. This pattern was observed 4 and 8 weeks following the first immunization.

It has been known that IgG1 and IgE are Th2-associated Ab, whereas IgG2a is a Th1-associated isotype Ab [Finkelman, F. D. et al., Ann. Rev. Immunol., 8: 303-333, 1990]. In particular, IgG2a production was significantly augmented by E7+ODN injection, as compared to E7 injection alone.

IgG2a/IgG1 was calculated as 0.2 (E7) and 0.26 (E7+ODN). This analysis suggests that ODN drives antigen-specific humoral immune responses overall in vivo.

### Example 4.2

Induction of Th Cell Proliferative Responses

Th cell proliferation is a standard parameter used to evaluate the potency of cell-mediated immunity. We measured Th cell proliferative responses following coimmunization with ODN by stimulating splenocytes from immunized animals in vitro with E7 proteins.

Th cell proliferation assay was performed as previously described [Sin, J. I. et al., Human Gene Therapy, 12: 1091-1102, 2001; Sin, J. I. et al., J. Immunol., 162: 2912-2921, 1999].

Each group of mice (n=4) was immunized s.c. with 20 μg of E7 and/or 20 μg of ODN at 0 and 2 weeks. Three weeks after the last immunization, spleen cells were obtained. The spleen cells were stimulated with E7 proteins at 0.5, 1 and 5 μg/ml concentrations for 3 days. Then, 3[H]-labeled thymidine (1 μCi per well) was added over night. Next day, the cells were harvested and cpm was counted using the β-counter (PerkinElmer, Boston, Mass.). Stimulation index (SI) was determined as ([experimental cpm-media control cpm]/[media control cpm]).

FIG. 4A represents the level of Th1 cell proliferative responses upon immunization with E7, CPG-ODN and E7 plus CpG-ODN. This was repeated 2 more times with similar results. *Statistically significant at p<0.05 using the paired Student's T test compared to negative controls. **Statistically significant at p<0.05 using the paired Student's T test compared to E7 alone.

As shown in FIG. 4A, E7 vaccination resulted in E7-specific Th cell proliferative responses. We also observed the significant enhancement of Th cell proliferative responses over that of E7 vaccine alone by immunization with E7+ODN. In contrast, the negative control group and the ODN immunized group showed little effects on the levels of Th cell proliferative responses.

This suggests that injection with E7 plus ODN can enhance E7-specific Th cell proliferative responses.

### Example 4.3

Induction of CTL Responses

A 5-h⁵¹Cr release assay was performed. Each group of mice (n=4) was immunized s.c. with 20 μg of E7 and/or 20 μg of ODN at 0 and 2 weeks. Three weeks after the last immunization, spleen cells were obtained. The splenocytes were stimulated for 5 days in the presence of 20 U/ml of IL-2 (R&D Systems, Minneapolis, Minn.) with TC-1 cells previously treated for 3 h with mitomycin C (30 μg/ml). TC-1 target cells were labeled with 100 μCi/ml Na251CrO4 for 2 h and used to incubate the stimulated splenocytes for 5 h at 37° C. One hundred μl of supernatants were harvested and counted on a gamma counter (Perkin Elmer). The percentage specific lysis was determined as 100×[(experimental release-spontaneous release)/(maximum release-spontaneous release)]. Maximum release was determined by lysis of target cells in 1% Triton X-100. An assay was not considered valid if the value for the spontaneous release counts was in excess of 20% of the maximum release value.

FIG. 4B represents the CTL induction levels in animals immunized with E7, CpG-ODN and E7 plus CpG-ODN. In this study, CTL was induced only in animal group immunized with both E7 and CpG-ODN. This was repeated two more times with similar results.

As demonstrated in FIG. 4B, only injection with E7 plus CPG-ODN induced CTL, suggesting that E7 in the presence of CPG-ODN can induce E7-specific CTL responses.

### Example 4.4

IFN-γ Production

IFN-γ plays an important role in inducing Th1 type immune responses as well as CTL responses. The levels of IFN-γ production from CD4+ and CD8+ T cells were measured. Each group of mice (n=4) was immunized s.c. with 20 μg of E7 and/or 20 μg of ODN at 0 and 2 weeks. Three weeks after the last immunization, spleen cells were obtained. 1 ml aliquot containing 6×106 splenocytes was added to wells of 24 well plates. Then, 1 μg of recombinant E7 protein/ml was added to each well. After 3 days incubation at 37° C. in 5% CO2, cell supernatants were secured and then used for detecting levels of IFN-γ using commercial cytokine kits (Biosource, Intl., Camarillo, Calif.) by adding the extracellular fluids to the IFN-γ-specific ELISA plates.

In more detail, splenocytes were stimulated in vitro with E7 protein to determine the production levels of IFN-γ from CD4+ T cells.

FIG. 5A represents the IFN-γ production levels of splenocytes upon stimulating the cells with 1 γg/ml E7 protein for 3 days. The spleen cells were obtained from animals immunized with E7, CPG-ODN, or E7 plus CpG-ODN. IFN-γ production was observed only in the animal group immunized with both E7 plus CpG-ODN. However, no production of IFN-γ was detected by immunization with either E7 or CpG-ODN alone. FIG. 5B represents the IFN-γ levels of splenocytes depleted of either CD4+ T or CD8+ T cells upon stimulation with 1 μg/ml E7 protein for 3 days. The spleen cells were obtained from animals co-immunized with E7 plus CpG-ODN. When splenocytes of E7+ODN immunized animals were depleted of CD4+ T cells, IFN-γ production was decreased to a background level, whereas CD8+ T cell depletion resulted in the same enhancement of IFN-γ production as whole splenocytes from E7+ODN injected animals. Values and bars represent the mean of released IFN-γ concentrations and the standard deviation. The experiments were repeated two more times with similar results.

This data suggests that E7 in the presence of CpG-ODN drives T cell responses predominantly in a Th1 type fashion and that CD4+ T cells are responsible for enhanced Th1 type cellular responses through the injection of E7+CpG-ODN. Furthermore, we also evaluated the CD8+ T cell dependent production level of IFN-γ. Splenocytes of animals immunized with E7 and/or ODN were subsequently stimulated in vitro with E7-expressing syngeneic TC-1 cells (MHC class I+, class II−) for 3 days.

FIG. 5C represents the IFN-γ levels of splenocytes upon stimulating with mitomycin C-treated TC-1 cells. The splenocytes were obtained from animals immunized with E7, CpG-ODN or E7 plus CpG-ODN. IFN-γ production was dramatically induced by injection with E7+ODN. However, little induction of IFN-γ production was observed in the groups injected with E7 or ODN alone. This is consistent with our previous observation that CTL (IFN-γ secreting CD8+ T cells) was induced only by E7+ODN coinjection (FIG. 4B).

FIG. 5D represents the IFN-γ levels of splenocytes depleted of CD4+ or CD8+ T cells upon stimulation with mitomycin C-treated TC-1 cells for 3 days. When splenocytes of E7+ODN immunized animals were depleted of CD8+ T cells, a background level of IFN-γ production was detected, in contrast to depletion of CD4+ T cells. Values and bars represent mean of released IFN-γ concentrations and the standard deviation. The experiments were repeated 2 more times with similar results.

This supports the notion that only injection of both E7 and CpG-ODN can induce IFN-γ production from CD8+ T cells in a MHC class I-dependent manner.

In the above, although the present invention was described by specific embodiments, a person skilled in this art may understand that any modifications and changes can be made within the spirit and scope of the invention.

What is claimed is:

1. A pharmaceutical composition for prophylaxis and therapy of cell proliferative diseases caused by papillomavirus, the pharmaceutical composition comprising:
an immunologically effective amount of papillomavirus E7 antigen protein and CpG-oligodeoxynucleotide.

2. The pharmaceutical composition of claim 1, wherein the papillomavirus E7 antigen protein is human papillomavirus type 16 E7 protein.

3. The pharmaceutical composition of claim 2, wherein the human papillomavirus type 16 E7 protein is a recombinant protein.

4. The pharmaceutical composition of claim 1, wherein the CpG-oligodeoxynucleotide comprises 8 to 40 nucleotides with one or more CpG motifs in which one or more nucleotides separate continuous CpG motifs in the oligodeoxynucleotide.

5. The pharmaceutical composition of claim 4, wherein CpG-oligodeoxynucleotide is 5′-TCCATGACGTTCCTGACGTT-3′.

6. The pharmaceutical composition of claim 1, wherein the disease caused by papillomavirus is cervical cancer.

7. The pharmaceutical composition of claim 2, wherein the disease caused by papillomavirus is cervical cancer.

8. The pharmaceutical composition of claim 3, wherein the disease caused by papillomavirus is cervical cancer.

9. The pharmaceutical composition of claim 4, wherein the disease caused by papillomavirus is cervical cancer.

10. The pharmaceutical composition of claim 5, wherein the disease caused by papillomavirus is cervical cancer.
